(19) United States
(12) Patent Application Publication
(10) Pub. No.: US 2015/0133983 A1
(43) Pub. Date: May 14, 2015

(54) HAND TOOL WITH REMOVABLE ATTACHMENT ASSEMBLY

(71) Applicant: Smith & Nephew, Inc., Memphis, TN (US)

(72) Inventor: Brian Loreth, Braintree, MA (US)

(21) Appl. No.: 14/533,458

(22) Filed: Nov. 5, 2014

Related U.S. Application Data
(60) Provisional application No. 61/904,134, filed on Nov. 14, 2013.

(51) Int. Cl. A61B 17/32; F16D 3/06; A61B 17/16

(52) U.S. Cl. CPC A61B 17/32002; A61B 17/1624; F16D 3/06; A61B 2017/00398
USPC 606/170; 29/428; 464/162

(57) ABSTRACT

A hand tool comprises: a handle, a cavity, and a drive adapter. The handle is disposed at a proximal end of the hand tool. The cavity is disposed at a distal end of the hand tool. The drive adapter is disposed in the cavity to rotate about an axis of the hand tool. In one embodiment, the drive adapter includes a hollowed volume extending through the drive adapter from a first opening at a tip of the drive adapter to a second opening (such as an a sidewall) of the drive adapter into the cavity. According to one configuration, the hollowed volume extends from the tip of the drive adapter to multiple openings disposed on a respective sidewall of the drive adapter. A respective sidewall of the drive adapter including one or more openings is free from contacting a circumferential surface in the hand tool defining the cavity.

### RELATED APPLICATIONS

This application is related to and claims the benefit of earlier filed U.S. Provisional Patent Application Ser. No. 61/904,134 entitled “HAND TOOL DRIVE UNIT,” (Attorney Docket No. SNI13-61(PT-4294-US-PSP)p), filed on Nov. 14, 2013, the entire teachings of which are incorporated herein by this reference.

This application is related to United States Patent Application entitled “HAND TOOL ATTACHMENT ASSEMBLY,” (Attorney Docket No. SNI13-62(PT-4293-US-NP), filed on the same day as this application, the entire teachings of which are incorporated herein by this reference.

### BACKGROUND

One type of conventional arthroscopic cutting tool includes a rigid, stationary outer tube within which a rigid inner tube rotates to perform a cutting operation. A cutting implement, such as a blade or abrading burr, is disposed on the distal end of the inner tube. As the inner tube rotates within the outer tube, a tip (at a distal end) of the cutting implement is applied to a surgical site. Matter such as tissue or bone fragments cut by the rotating blade at the cutting tip are drawn through the interior of the inner tube along with irrigating fluid via suction applied to the inner tube.

An example of a conventional cutting drive assembly 200 for use in surgical applications is shown in FIG. 1. In general, to use the cutter drive assembly 200, a proximal end of the cutter drive assembly 200 is engaged into a motorized hand tool. During operation, the motorized hand tool rotates the inner tube within the outer tube to provide cutting capability at cutting tip 220. Details of an example conventional surgical instrument using cutter assembly 200 are further discussed in United States patent number 5,601,583 issued to Donahue, et al.

### BRIEF DESCRIPTION

A drawback of the conventional cutter tool as shown in FIG. 1 is that the complex and expensive-to-manufacture drive portion at proximal end of assembly 200 is thrown away with the cutter blade portion after use. Thus, use of assembly 200 to perform cutting operations can be costly.

In contrast to conventional cutter tools, embodiments herein include drive adapter comprising: a first opening, a second opening, and a base. The drive adapter as discussed herein can be configured for installation in a respective hand tool. The first opening is disposed at a distal end of the drive adapter. The second opening is disposed on a sidewall of the drive adapter. A hollowed volume in the drive adapter extends between the first opening and the second opening. The hollowed volume facilitates a flow of matter (such as solid, liquid, and/or gas) into the first opening and out the second opening. The base end (e.g., disposed at a proximal end opposite the tip including the first opening) of the drive adapter is fabricated to securely couple the drive adapter to a drive component (such as a drive shaft, drive source, etc.) in a respective hand tool.

In accordance with further embodiments, the drive adapter includes a keyed region disposed along a portion its axial length. The keyed region is configured to engage the drive adapter to a respective drive component. In one embodiment, the keyed region of the drive adapter is located along a portion of the axial length of the drive adapter between the hollowed volume and the proximal end of the drive adapter. In one embodiment, the keyed region of the drive adapter facilitates axial sliding of the base end of the drive adapter in a respective drive component into which the base end of the drive adapter is inserted. The keyed region of the drive adapter matably connects to the drive component such that the drive adapter and the drive component rotate in unison.

In accordance with further embodiments, the first opening at the distal end of the drive adapter includes a keyed interface to accept a tube of a blade assembly. Rotation of the drive adapter causes the tube of the blade assembly to rotate.

Further embodiments herein include a notch or other suitable resource disposed on the drive adapter. The notch and corresponding retainer resource anchors the base end of the drive adapter to the drive component.

Further embodiments herein include a hand tool comprising: a handle, a cavity, and a drive adapter. The handle is disposed at a proximal end of the hand tool. The cavity is disposed at a distal end of the hand tool. The drive adapter is disposed in the cavity to rotate about an axis of the hand tool. In one embodiment, as previously discussed, the drive adapter includes a hollowed volume extending through the drive adapter from a first opening at a tip of the drive adapter to one or more additional openings (such as on a sidewall) of the drive adapter into the cavity.

In accordance with further embodiments, the hollowed volume extends from the tip of the drive adapter to the one or more openings disposed on a respective sidewall of the drive adapter. The respective sidewall of the drive adapter including the one or more openings is free from contacting a circumferential surface in the hand tool defining the cavity.

The drive adapter includes a base end disposed opposite the end of the drive adapter including the tip; the base end of the drive adapter is coupled to a rotatable drive component in the hand tool. During operation, the drive component applies a rotational force to the base end of the drive adapter to rotate the drive adapter about an axis.

In accordance with further embodiments, the drive adapter is spring-loaded in the drive component to permit slidable movement of the drive adapter along the axis. Accordingly, application of force on the tip of the drive adapter causes the drive adapter to move deeper within the cavity of the hand tool; release of the application of force on the tip causes the drive adapter to move in an outward direction from the cavity until the drive adapter reaches a stop, preventing the drive adapter from being pulled out of the cavity.

In further embodiments, the hand tool further includes a drive component such as a drive shaft. The drive adapter is coupled to a drive source (such as a motor or engine) via the drive component. During operation, the drive source rotates the drive component about an axis; the drive adapter is anchored or secured to the drive component to prevent removal of the drive adapter from the drive component and the cavity.

The first opening at the tip of the drive adapter can be configured to include a keyed interface in which to engage a tube of a rotatable blade assembly to the drive adapter. During operation, the drive adapter transfers torque generated by the drive source to the tube disposed in the keyed interface.

In accordance with yet further embodiments, the hollowed volume of the drive adapter includes a first hollowed volume portion and a second hollowed volume portion. The first hollowed volume portion extends along the axis from the first opening at the tip of the drive adapter to the second hollowed volume portion on the sidewall of the drive adapter. The second hollowed volume portion extends through the drive adapter orthogonal or substantially orthogonal with respect to the axis of rotation of the drive adapter.

The hand tool can be configured further to include a port. The port enables application of suction to the cavity; the suction draws matter through a fluid pathway including: i) a core of a tube inserted into the tip of the drive adapter, ii) the first hollowed volume portion of the drive adapter, iii) the second hollowed volume portion of the drive adapter, iv) the cavity at the distal end of the hand tool, and v) the port.

In still further embodiments, the hand tool includes a seal disposed between a circumferential outer surface of the drive component and a body of the hand tool. The seal prevents exposure of contaminants present in the cavity to a bearing resource in the hand tool facilitating rotation of the drive component.

As further discussed herein, cutting system can include the hand tool and a corresponding attachable blade assembly. The blade assembly securely attaches to the distal end of the hand tool including the cavity in which the drive adapter resides. During operation, the drive adapter coupled to an inner tube of the blade assembly provides torque to spin the inner tube of the attachable blade assembly.

Further embodiments herein include fabrication of a respective hand tool including the drive adapter as previously discussed. For example, fabrication resource receives a drive adapter. As previously discussed, the drive adapter includes a (hollowed) tip end and a base end. The drive adapter includes a hollowed volume extending from the first opening at the tip end through the drive adapter to a second opening on a sidewall of the drive adapter.

The fabrication resource inserts the drive adapter into a cavity at a distal end of the hand tool. Additionally, the more specifically, the fabrication resource inserts the base end of the drive adapter into a cavity of a drive component of the hand tool.

In accordance with further embodiments, a spring disposed in the cavity of the drive component applies a force to the base end of the drive adapter, pushing the base end of the drive adapter away from the drive component.

The fabrication resource further anchors the base end of the drive adapter within the cavity of the drive component. The tip of the drive adapter (opposite the base at the distal end of the drive adapter) resides within a cavity of the hand tool. The sidewall of the drive adapter is rotatably free from contacting an internal surface of the cavity.

Embodiments herein are useful over conventional surgical instruments for a number of reasons. For example, the hand tool assembly as described herein is substantially lower in complexity than conventional cutter blade assemblies. Lower complexity results in benefits such as lower fabrication costs, ease of attaching the blade assembly to a corresponding hand tool, etc.

These and other more specific embodiments are disclosed in more detail below.

As discussed herein, techniques herein are well suited for use in cutting applications. However, it should be noted that embodiments herein are not limited to use in such applications and that the techniques discussed herein are well suited for other applications as well.

Additionally, note that although each of the different features, techniques, configurations, etc., herein may be discussed in different places of this disclosure, it is intended, where suitable, that each of the concepts can optionally be executed independently of each other or in combination with each other. Accordingly, the one or more present inventions as described herein can be embodied and viewed in many different ways.

Also, note that this preliminary discussion of embodiments herein purposefully does not specify every embodiment and/or incrementally novel aspect of the present disclosure or claimed invention(s). Instead, this brief description only presents general embodiments and corresponding points of novelty over conventional techniques. For additional summary and details and/or possible perspectives (permutations) of the invention(s), the reader is directed to the Detailed Description section and corresponding figures of the present disclosure as further discussed below.

### BRIEF DESCRIPTION OF THE DRAWINGS

The foregoing and other objects, features, and advantages of the invention will be apparent from the following more particular description of preferred embodiments herein, as illustrated in the accompanying drawings in which like reference characters refer to the same parts throughout the different views. The drawings are not necessarily to scale, with emphasis instead being placed upon illustrating the embodiments, principles, concepts, etc.

FIG. 1 is an example perspective image of a cutter assembly according to conventional techniques.

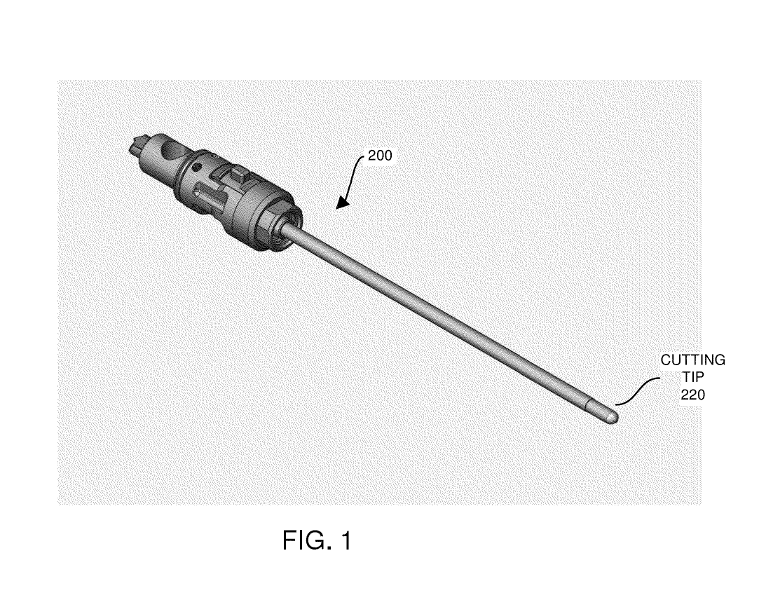

FIG. 2 is an example perspective view diagram of a cutter blade assembly selectively coupled to a hand tool according to embodiments herein.

FIG. 3 is an example side view diagram of a distal end of a cutter blade assembly according to embodiments herein.

FIG. 4 is an example side view diagram of a cutter blade assembly coupled to a drive unit according to embodiments herein.

FIG. 5 is an example perspective view diagram of a cutter blade assembly coupled to a drive unit according to embodiments herein.

FIG. 6 is an example cutaway side view diagram of a hand tool and corresponding drive unit according to embodiments herein.

FIG. 7 is an example cutaway perspective view diagram of a driver adapter according to embodiments herein.

FIG. 8 is an example perspective view diagram of a drive adapter according to embodiments herein.

FIG. 9 is an example diagram illustrating spring loading of a driver adapter according to embodiments herein.

FIG. 10 is an example perspective diagram of a cutter system according to embodiments herein.

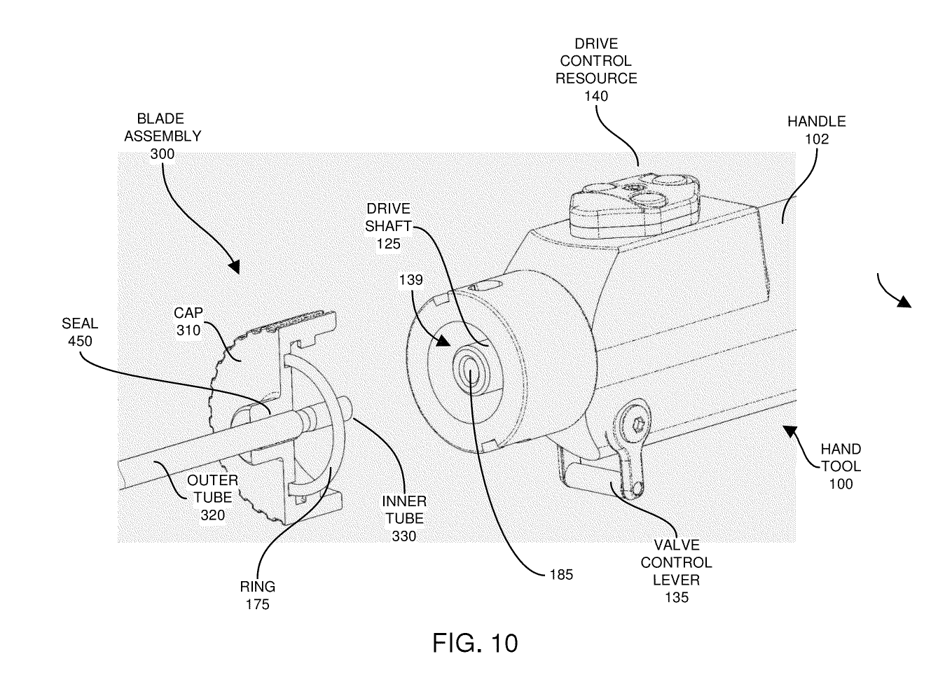

FIG. 11 is a method of fabricating a hand tool according to embodiments herein.

FIG. 12 is an example cutaway perspective view diagram illustrating use of a respective retainer resource to secure a drive adapter to a driveshaft assembly according to embodiments herein.

### DETAILED DESCRIPTION

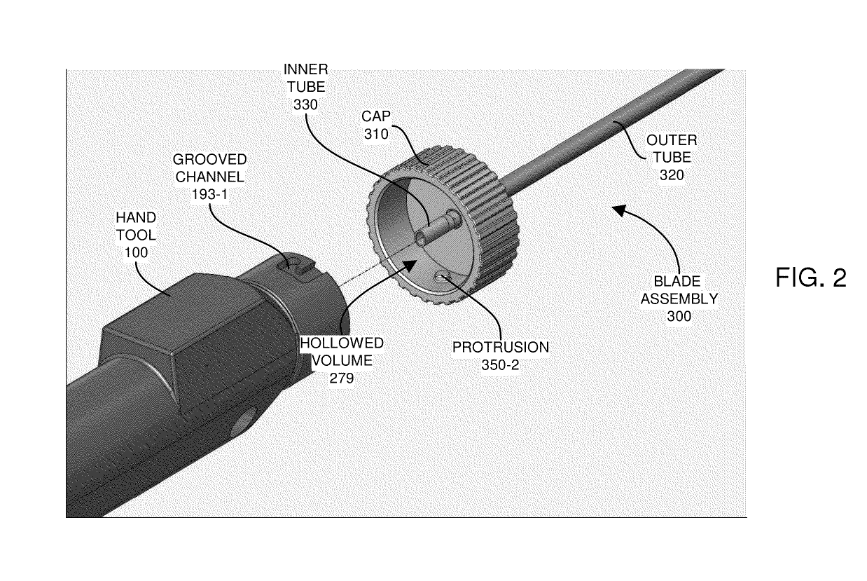

Now, more specifically, FIG. 2 is an example perspective diagram illustrating a proposed blade assembly 300 according to embodiments herein. As shown, blade assembly 300 includes cap 310, inner tube 330, and outer tube 320.

Cap 310 includes hollowed volume 279 for coupling the blade assembly 300 to the distal end of hand tool 100.

If desired, cap 310 can include any suitable type of fastener resource such as grooved regions, threads, etc., facilitating twisting and securing of the cap 310 to a respective distal end of hand tool 100. In one non-limiting example embodiment, the hand tool 100 includes one or more channels including grooved channel 193-1. Cap 310 includes one or more protrusions such as protrusion 350-2 that secure cap 310 to the distal end of hand tool 100. As further discussed below, use of protrusions and grooved channels to secure the 310 to the distal end of hand tool 100 is shown by way of non-limiting example only. Any suitable technique can be used to secure cap 310 to the distal end of hand tool 100.

Note that each of the components in FIG. 2 such as cap 310, inner tube 330, and outer tube 320 can be fabricated from any suitable type of material such as metal, plastic, etc.

In one embodiment, inner tube 330 slides and rotates within outer tube 320 and is removable. Inner tube 330 is made of substantially rigid material or semi-rigid material and extends at least a length of outer tube 320. A proximal end (neuron) of outer tube 320 is fixedly attached to cap 310.

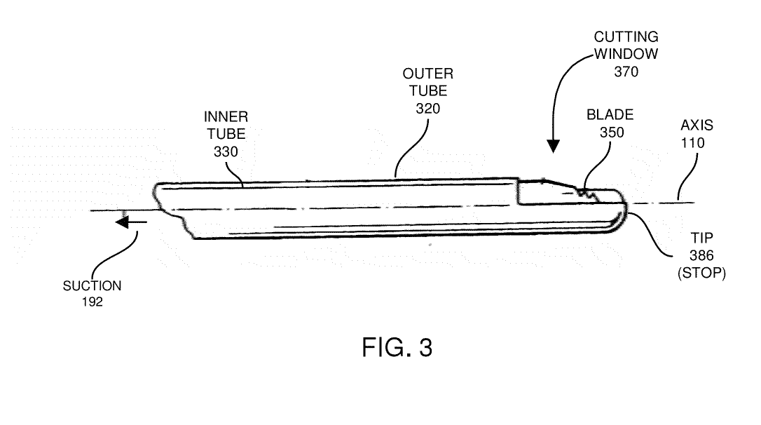

As further shown in FIG. 3, by further way of a non-limiting example, distal end of outer tube 320 includes a respective cutting window 370. In one embodiment, distal end of the inner tube 330 includes a respective blade 350 (potentially with serrated teeth, razor-edge, etc.) and corresponding cutting window that cuts tissue residing within cutting window 370 of outer tube 320.

During operation, hand tool 100 controls application of suction 192 to inner tube 330. Suction 192 applied to inner tube 330 causes loose matter in a vicinity of the spinning blade 350 at distal end of inner tube 330 to be drawn through cutting window 370 at the distal end of tube 320 and respective opening (a rotating second cutting window) at the distal end of the inner tube 330 towards hand tool 100.

Referring again to FIG. 2, in this non-limiting example embodiment, cap 310 selectively connects to distal end of hand tool 100 including one or more grooved channels 193. For example, one or more protrusions 350 such as protrusion 350-2 on inside circumferential surface of cap 310 slides into a respective grooved channel 193-2. After sliding, a twist of cap 310 secures the cap 310 to distal end of hand tool 100.

Note that as an alternative to a grooved channel/protrusion locking mechanism, any suitable type of means can be used to secure the cap 310 to the distal end of the hand tool 100. For example, as an alternative to a J-groove locking mechanism, an outer circumference of the distal end of the hand tool 100 can be configured to include threads. Corresponding threads located on the inner circumferential wall of cap 310 can be configured to accept threads on the inner circumferential surface of the cap 310. In such an instance, the cap 310 can be screwed onto the distal end of hand tool 100.

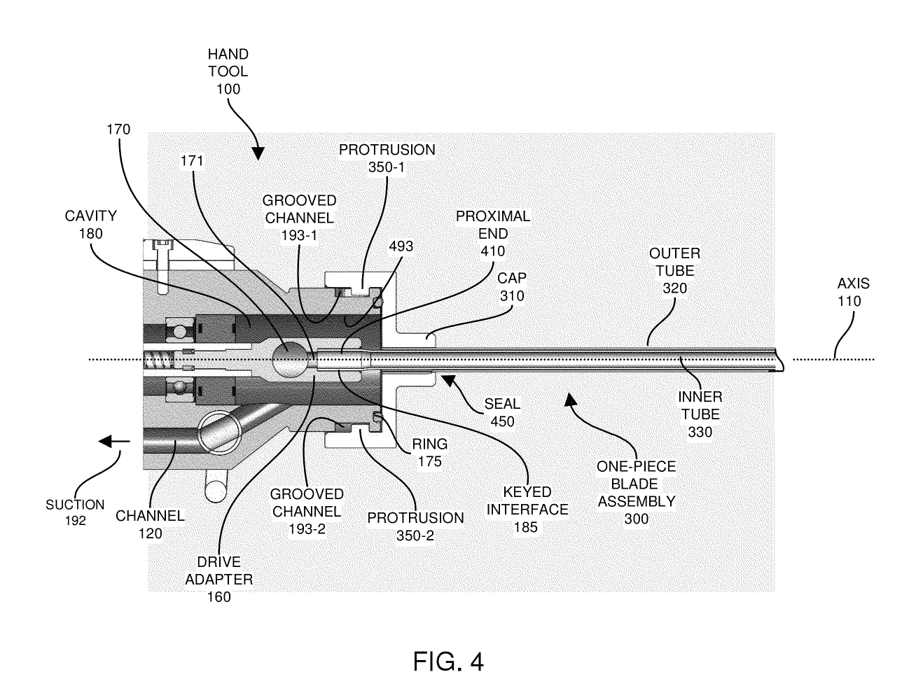

FIG. 4 is an example cross-sectional view diagram illustrating a proposed one-piece blade adapter attached to a corresponding hand tool according to embodiments herein.

As shown, and as previously discussed, cap 310 couples to distal end of hand tool 100. Protrusions 350 (such as protrusions 350-1, 350-2, etc.) slide into respective grooved channels 193 (grooved channel 193-1, grooved channel 193-2, etc.) to lock cap 310 in place onto distal end of hand tool 100. More specifically, protrusion 350-1 slides in grooved channel 193-1; protrusion 350-2 slides in grooved channel 193-2; etc. Subsequent to rotating the cap 310 to secure cap 310 to the distal end of the hand tool 100, ring 175 and seal 450. The seal 450 can be based on a fluid-tight fit created (such as via overmolding, heat staking, etc.) between outer circumferential surface of the outer tube 320 and respective inner circumferential surface of an opening disposed in cap 310 as shown. If desired, seal 450 can include material such as glue, silicone, etc. Seal 450 ensures that cavity 180 is fluid-tight with respect to surrounding environment outside of cavity 180. As will be discussed in more detail later in the specification, suction 192 applied to channel 120 and respective cavity 180 causes matter received in a respective window at distal end of outer tube 320 to flow through a path including: inner tube 330, proximal end 410, keyed interface 185, hollowed volume 171, hollowed volume 170, into and through cavity 180 to channel 120.

During operation, the distal end of drive adapter 160 rotates about respective axis 110. The outer surface of the drive adapter 160 is free from contacting corresponding outer circumferential surface 493 in the cavity 180. In other words, the outer surface of the drive adapter 160 does not contact the surface 493 in cavity 180 such that the drive adapter 160 is free to rotate without friction.

Further, proximal end 410 of inner tube 330 matably attaches to keyed interface 185. Thus, rotation of the drive assembly 160 about axis 110 causes the inner tube 330 to rotate about axis 110. As previously discussed, inner tube 330 rotates with respect to outer tube 320.

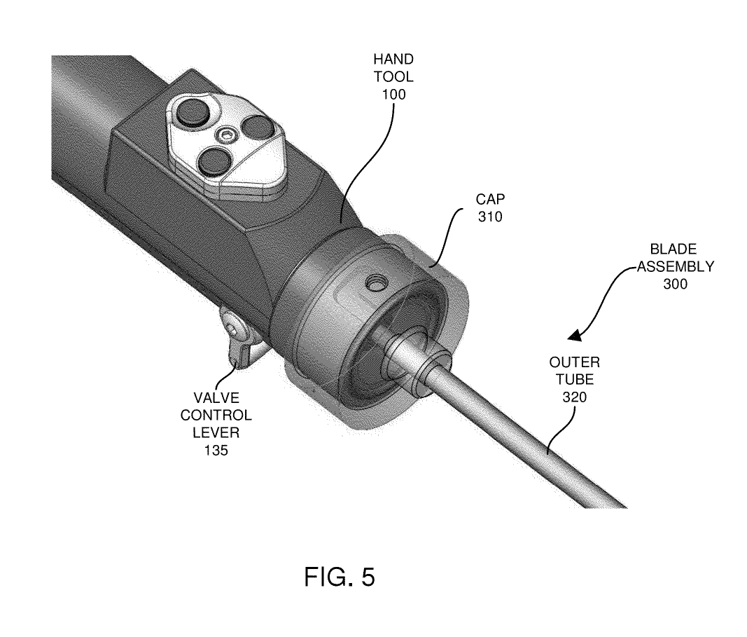

FIG. 5 is an example perspective diagram illustrating attachment of the blade assembly 300 to the distal end of hand tool 100 according to embodiments herein. As further discussed below, valve control lever 135 controls an amount of suction 195 applied to the inner tube 330.

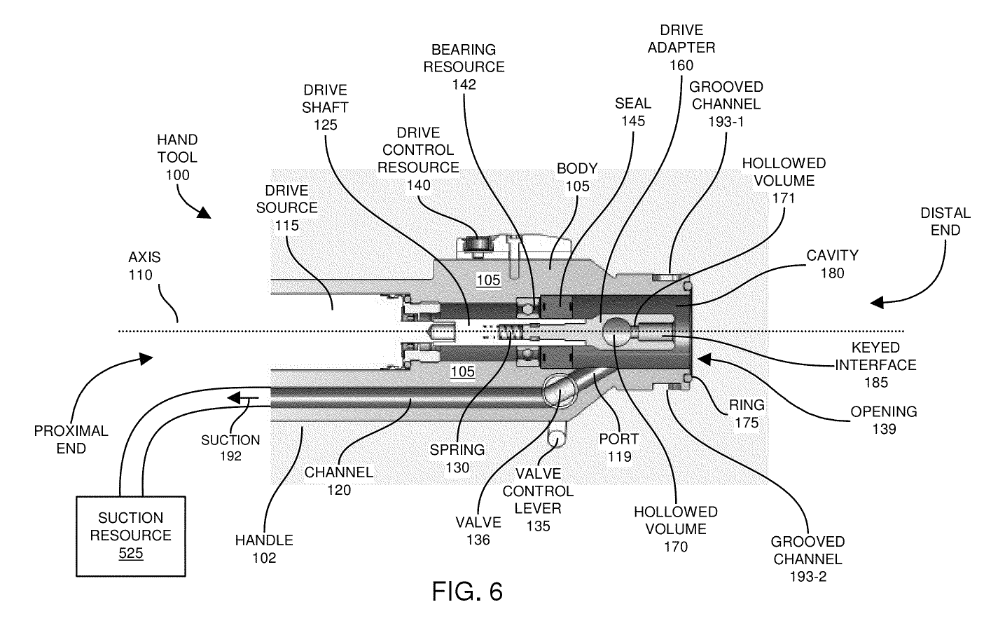

FIG. 6 is an example diagram illustrating a more detailed version of a proposed drive source according to embodiments herein.

As shown, hand tool 100 includes a handle portion 102 disposed at the proximal end of hand tool 100 for gripping by a respective user such as a surgeon. When the respective cap 310 of the assembly element 300 is securely attached to the distal end of hand tool 100, the user gripping handle portion 102 of hand tool 100 is able to steer the corresponding tip 386 (and corresponding cutting window 370) to a respective location such as a surgical site.

As further shown, drive shaft 125 (i.e., a drive component) is matably attached to drive source 115 in hand tool 100. Activation of the drive source 115 (such as a spinning electric motor, pneumatic motor, etc.) causes rotation of drive shaft 125 about axis 110.

Note that the drive shaft 125 can be fabricated from any suitable one or more types of material such as hardened stainless steel, plastic, epoxy, etc.

Bearing resource 142 disposed in body 105 of hand tool 100 facilitates rotation of drive shaft 125 with respect to (substantially stationary) body 105 of hand tool 100. Presence of seal 145 prevents debris in cavity 180 from passing to bearing resource 142.

Additionally, hand tool 100 includes drive control resource 140. The user operating hand tool 100 controls the drive control resource 140 (such as one or more buttons, triggers, etc.) to selectively activate rotation of the drive shaft 125 (i.e., a drive component).

As a specific example, pressing a respective button of drive control resource 140 to an ON position causes the drive source 115 to spin drive shaft 125 about axis 110. Releasing the button of drive control resource 140 to an OFF position causes the drive resource 115 to cease spinning of the drive shaft 125 about axis 110.

If desired, the drive control resource 140 can be configured to one or more speed buttons to control the speed of rotating the drive adapter 160 and corresponding inner tube 330.

As further shown, distal end of hand tool 100 includes cavity 180 (such as hollowed volume or housing) in which drive adapter 160 resides. Drive adapter 160 (such as made from stainless steel material or other suitable material) is mechanically coupled to a distal end of drive shaft 125 such that rotation of the drive shaft 125 (and drive source 115) causes rotation of drive assembly 160 as well. As previously discussed, the proximal end 410 of the inner tube 330 can be configured to reside in keyed interface 185 upon securing of cap 310 to the distal end of hand tool 100. Rotation of the drive shaft 125 causes rotation of drive adapter 160; rotation of drive adapter 160 causes rotation of inner tube 330.

Accordingly, rotational forces produced by drive source 115 translate through drive shaft 125 and drive adapter 160 to inner tube 330.

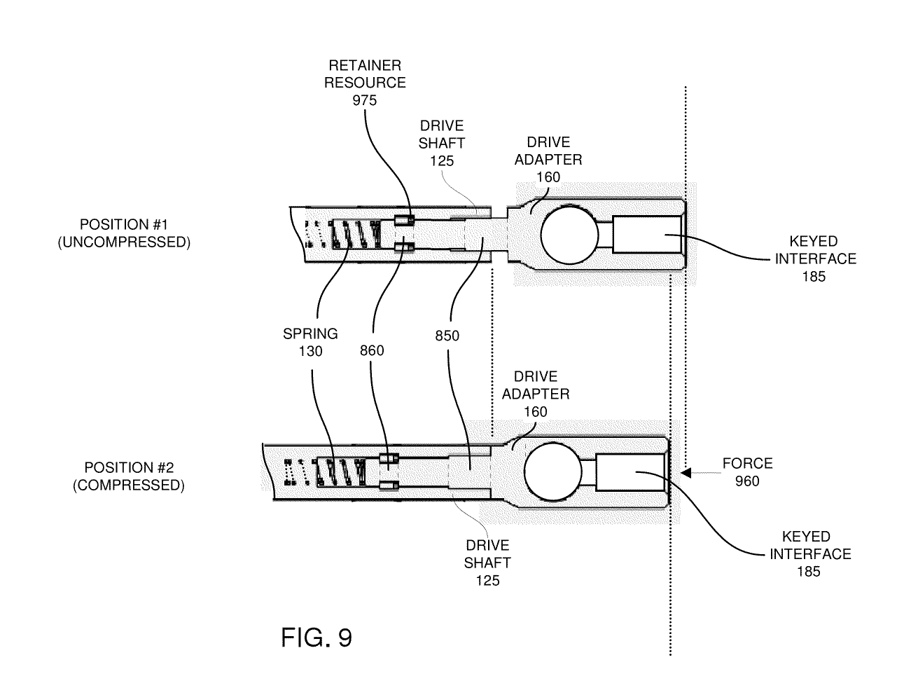

In one embodiment, a corresponding end of the drive adapter 160 (e.g., in and near the drive source 115) slidably moves in a hollowed volume (keyed-bore) of drive shaft 125 along axis 110 with respect to drive shaft 125. Thus, the drive adapter 160 slides within a respective bore (receptacle) at a distal end of the drive shaft 125. When the drive assembly 160 is pushed inward towards drive source 115 via application of a respective force substantially along axis 110, spring 130 compresses (a state as shown). When the force applied to the drive adapter 160 is removed, spring 130 decompresses and pushes drive assembly 160 outward again to a corresponding resting position in cavity 180. (This operation is further illustrated in FIG. 9 and corresponding text.)

Thus, in addition to the drive adapter 160 spinning about axis 110 along with drive shaft 125, the drive assembly 160 movably slides in cavity 180 along axis 110 depending on application of a corresponding force applied to the distal end of drive adapter 160.

Additionally, drive adapter 160 can be anchored in any suitable manner (such as via a retaining ring, clip, etc.) to the drive shaft 125 so that it does not release (or easily release) from drive shaft 125 based on application of force to the drive adapter 160 along axis 110 to pull the drive adapter 160 out of cavity 180. Further, as previously discussed, the proximal and 410 of the inner tube 330 resides in keyed interface 185 of the drive adapter 160 when the cap 310 is secured to the distal end of hand tool 100 at opening 139. Anchoring of the drive adapter 160 to the drive shaft 125 ensures that when the proximal and 410 of the inner tube 330 is removed from (pulled out of) the keyed interface 185, the drive adapter 160 is not pulled free from the drive shaft 125 out of cavity 180 (i.e., an open or closed hollowed volume depending upon whether cap 310 is secured to the distal end of the hand tool 100).

In certain instances, however, the drive adapter 160 can be configured to be removable from the distal end of drive shaft 125 and cavity 180 for maintenance. Yet further in these non-limiting example embodiments, cavity 180 includes seal 145 (such as made from polymer material or other suitable materials as part of a sealing system or assembly) to prevent matter such as fluid, debris, etc., disposed in cavity 180 from contaminating bearing resource 142, drive source 115, etc.

For example, an outer circumferential surface of seal 145 contacts a corresponding inner circumferential surface of cavity 180 disposed in body 105 of hand tool 100; an inner circumferential surface of seal 145 contacts a corresponding outer surface of drive shaft 125. There is relatively low friction between the inner compass circumferential surface of seal 145 and the corresponding outer circumferential surface of drive shaft 125. The tightness of fitting seal 145 with respect to body 105 and drive shaft 125 enables unimpeded rotation of drive shaft 125 and corresponding drive adapter 160, but prevents contaminants in cavity 180 from passing through seal 145 to bearing resource 142, drive source 115, etc.

Thus, via seal 145, drive source 115 and bearing resource 142 are not exposed to contaminants that may be presence or passing through cavity 180.

Channel 120 (such as a tubular region or lumen) extends from port 119 of cavity 180 disposed at distal end of hand tool 100 to a proximal end of hand tool 100 where suction 192 is applied via activation of suction resource 525. In other words, suction resource 525 can be configured to selectively produce a vacuum or negative pressure, resulting in suction 192 of matter through channel 120 towards suction resource 525.

In this non-limiting example embodiment, the valve control lever 135 controls a position of corresponding valve 136 and corresponding flow from cavity 180 through channel 120 to suction resource 525. For example, opening of valve 136 enables suction 192 generated by suction resource 525 to pull matter (such as solids, liquids, and gases) in cavity 180 through port 119 into and through channel 120.

Note further that a distal end of drive assembly 160 includes hollowed volume 170 (such as a radial bore, an axis of which is disposed orthogonal or substantially orthogonal with respect to axis 110). Hollowed volume 171 (an axial bore) extends along axis 110 from distal end of the drive assembly 160 to hollowed volume 170.

The combination of hollowed volume 170 and hollowed volume 171 provides a hollowed passageway extending from an opening (such as keyed interface 185) at distal end of drive adapter 160 into cavity 180. As previously discussed, when suction 192 is applied to channel 120 when valve 136 is OPEN, suction 192 through channel 120 and port 119 provides passage of matter in cavity 180 to proximal end of hand tool 100. Thus, port 119 enables application of suction to cavity 180.

Referring again to FIG. 4, the keyed interface 185 at distal end of drive adapter 160 accepts proximal end 410 of inner tube 330 associated with blade assembly 300. The keyed interface 185 ensures that the corresponding inner tube 330 rotates about axis 110 when the corresponding drive source 115 is activated via drive control resource 140. As previously discussed, rotation or spinning of the inner tube 330 about axis 110 (in accordance with drive source 115) enables distal end of inner tube 330 exposed in cutting window 370 to cut matter such as tissue.

Because the inside of inner tube 330 is hollow, the suction 192 applied to cavity 180 causes matter (such as a solid, liquid, or gas material) passing through cutting window 370 to be sucked through a fluid pathway including inner tube 330, hollowed volume 171, and hollowed volume 170 to cavity 180 (such as a fluid-tight chamber). As previously discussed, matter in cavity 180 is sucked through channel 120 to suction resource 525 via application of suction 192.

Further note that a distal end of the hand tool 100 can include a sealing resource such as a ring 175 (fabricated from rubber or other suitable elastic material) and corresponding grooved channels 193 (such as grooved channel 193-1 and grooved channel 193-2) to lock and seal a respective cap portion of the rotatable blade assembly to the distal end of hand tool 100. Inclusion of the ring 175 ensures that the cavity 180 becomes a fluid-tight when the respective cap 310 is coupled to cover opening 139 at the distal end of hand tool 100. That is, ring 175 prevents leakage of air into cavity 180 through respective sidewalls of capacitor 310 and a respective outer surface of distal end of the hand tool 100. As previously discussed, attachment of the cap 310 to the distal end of hand tool 100 can be achieved in any suitable manner. For example, the cap 310 can be secured to the distal end of hand tool 100 via threads, J-locks, etc.

As an alternative to including ring 175 in a groove at the distal end of the hand tool 100, embodiments herein can further include disposing a respective a seal resource (such as a gasket, O-ring, etc.) on a facing of the cap 310 in the hollowed volume 279. Presence of the gasket in cap 310 ensures that the cavity 180 is fluid-tight.

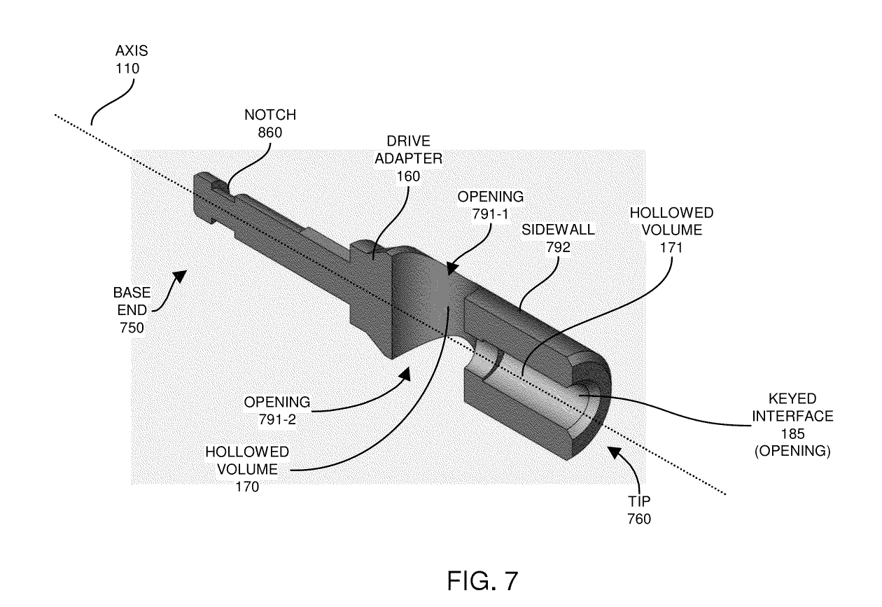

FIG. 7 is an example cutaway perspective view diagram of a driver adapter according to embodiments herein.

As shown in this cutaway perspective view diagram, the drive adapter 160 includes hollowed volume 170 and hollowed volume 171. By way of non-limiting example embodiment, the hollowed volume 171 of drive adapter 160 includes keyed interface 185 (such as an opening) at tip 760, which accepts proximal end 410 of the inner tube 330.

Alternatively, note that the keyed interface 185 can be disposed on an outer circumferential surface at a tip of the drive adapter 160 as opposed to being a cavity or opening at the distal end of the drive adapter. In such an instance, the keyed interface 185 disposed on the outer surface of the drive adapter 160 fits into a matching keyed opening at the proximal end 410 of the inner tube 330. In other words, in this latter embodiment, the opening at the proximal and 410 of the inner tube 330 acceptably receives the keyed interface 185 disposed at the distal end of drive adapter 160.

Accordingly, the keyed interface 185 can be located on the internal or external surface of the drive adapter 160 depending on the embodiment.

As previously discussed, inclusion of the keyed interface 185 ensures that the inner tube 330 and drive adapter 160 rotate in unison about axis 110 when the drive adapter 160 is rotated about axis 110.

As further shown, the hollowed volume 170 in the drive adapter extends to the sidewall 792 of the drive adapter 180, creating openings 791-1 and opening 791-2. As further discussed in the following FIGS., the base end 750 of the drive adapter 180 includes notch 860 to accept a retainer resource such as a retaining ring, spring clip, etc., preventing the drive adapter 160 from being pulled out of the drive shaft 125 and corresponding cavity 108.

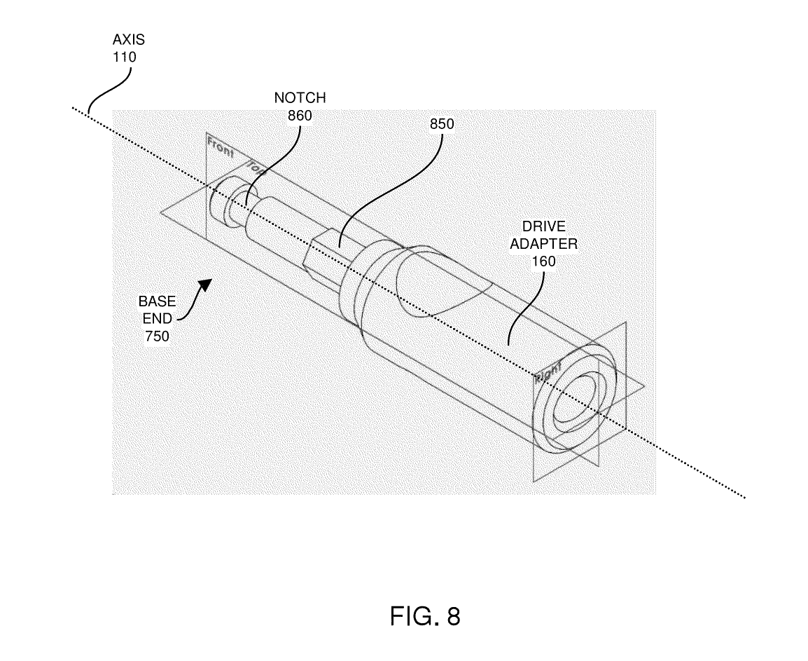

FIG. 8 is an example perspective view diagram of a drive adapter according to embodiments herein.

As shown in this example embodiment, drive adapter 160 includes keyed region 850 (such as a hexagonal bolt pattern or other suitable pattern) that fits into a corresponding hollowed portion at a distal end of drive shaft 125 as previously discussed.

The keyed (but axially slidable) fit between the drive shaft 125 and keyed region 850 of the drive adapter 160 ensures that the drive shaft 125 and drive adapter 160 spin together about axis 110 when a corresponding rotational force is applied to the drive shaft 125.

Additionally, drive adapter 160 includes one or more a holding mechanism such as a notch 860 to accept the retaining ring or spring clip to secure or anchor the driver adapter 160 within drive shaft 125. In one embodiment, the anchor enables the base end 750 of the drive adapter 160 to slide within a corresponding cavity of the drive shaft 125. Anchoring or securing of the drive adapter 160 to the drive shaft 125 ensures that the drive adapter 160 is not easily pulled free from the drive shaft 125 when the corresponding proximal end 410 of the inner tube 330 is removed from the keyed interface 185 of the drive adapter 160.

In accordance with further embodiments, the drive adapter 160 is removable from the cavity 180 to facilitate cleaning of matter from the cavity 180.

In contrast to conventional techniques, the drive adapter 160 can be reused to perform cutting operations because it is not disposed of with a corresponding blade assembly.

FIG. 9 is an example diagram illustrating spring loading of a driver adapter according to embodiments herein.

As shown, in an uncompressed position #1, the spring 130 applies a force to drive adapter 160, pushing drive adapter 160 to an extended position. One or more anchors such as retainer resource 975 in notch resource 860 prevents the drive assembly 160 from being pulled away from driveshaft 125.

As further shown in position #2, application of force 960 to the drive adapter 160 causes the spring 130 to compress and movement of the drive assembly 160 into driveshaft 125.

Whether in the compressed or uncompressed position, the insertion of keyed region 850 (slidable within the driveshaft 125 as previously discussed) into a corresponding cavity of the driveshaft 125 ensures that the drive adapter 160 rotates along with driveshaft 125 when drive source 115 applies a corresponding torque to driveshaft 125.

By way of non-limiting example embodiment, the spring 130 can be configured to exert a force 960 between 0.1 and 5 pounds to the drive adapter 160. However, note that the spring 130 (or other suitable compressible resource) can be configured to apply any suitable compression force to drive adapter 160. Thus, the amount of force 960 needed to compress the spring 130 can vary depending upon the embodiment.

As previously discussed, the force 960 provided by spring 130 ensures that the blade 350 at the distal end of inner tube 330 is pushed up against tip 386 disposed on the distal end of outer tube 320.

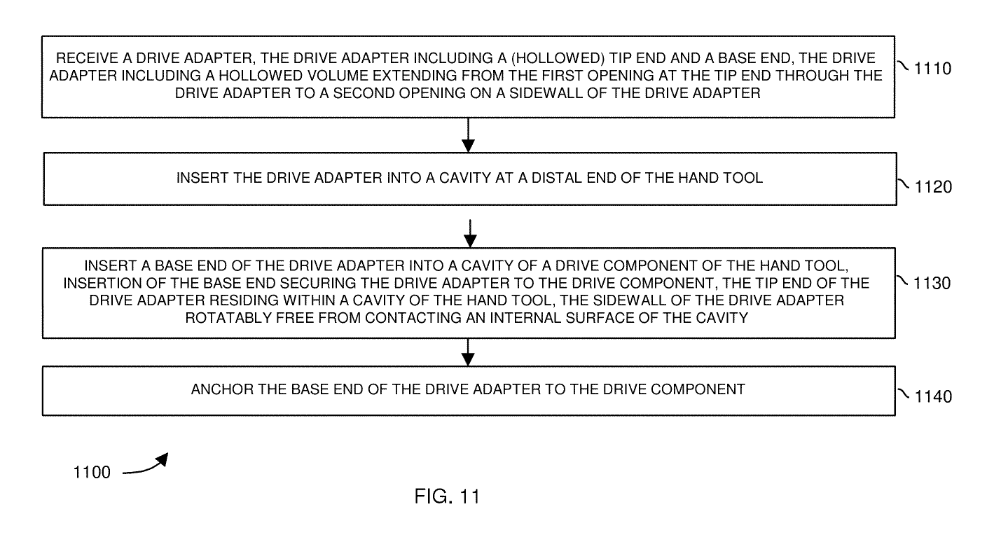

FIG. 11 is a flowchart 1100 illustrating an example method according to embodiments. Note that there will be some overlap with respect to concepts as discussed above.

In processing block 1110, a fabrication resource (such as a manufacturing facility, assembly-line worker, automated assembler, etc.) receives a drive adapter 160. The drive adapter 160 includes a (hollowed) tip 760 and a base end 750. The drive adapter 160 includes a hollowed volume 170 and 171 extending from the first opening (keyed interface 185) at the tip 760 through the drive adapter 160 to a second opening (such as opening 791-1, 791-2, etc.) disposed on a sidewall 792 of the drive adapter 160.

In processing block 1120, the fabrication resource inserts the drive adapter 160 into a cavity 180 disposed at a distal end of the hand tool 100.

In processing block 1130, the fabrication resource inserts a base end 750 of the drive adapter 160 into a cavity of a drive component (e.g., into driveshaft 125) of the hand tool 100. Insertion of the base end 750 secures the drive adapter 160 to the drive component (driveshaft 125 or drive source 115) with a retainer resource 975 such as a ring, clip, or other suitable resource. Subsequent to insertion, the tip 760 of the drive adapter 160 resides within the cavity 180 of the hand tool 100. The sidewall of the drive adapter 160 is rotatably free from contacting an internal surface 493 of the cavity 180.

In processing block 1140, the fabrication resource anchors the base end 750 of the drive adapter 160 to the drive component utilizing retainer resource 975.

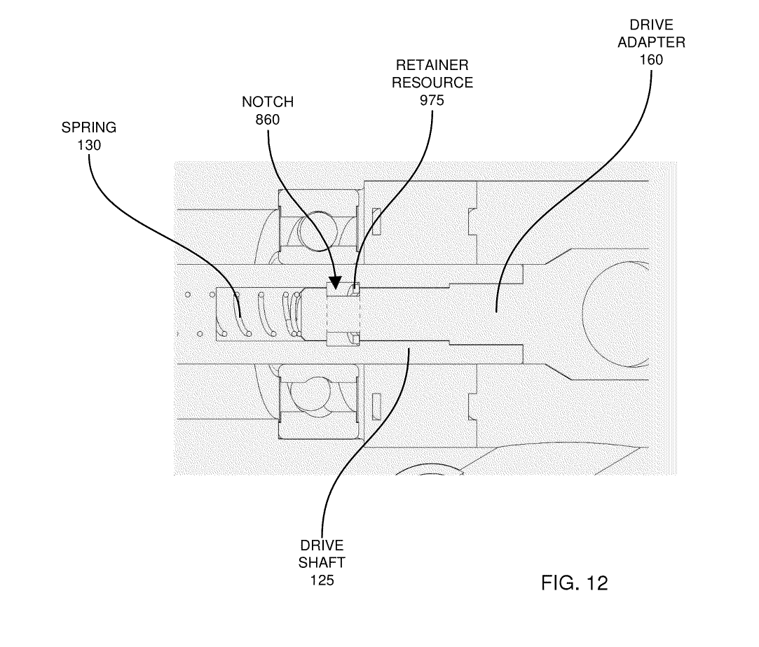

FIG. 12 is an example cutaway perspective view diagram illustrating use of a respective retainer resource to securely attach a drive adapter to a driveshaft assembly according to embodiments herein.

Specifically, as shown, retainer resource 975 disposed in notch 860 secures the drive adapter 160 to the drive shaft 125. As previously discussed, the retainer resource 975 enables the notched end of drive adapter 160 (i.e., an end of the drive adapter 160 including notch 860) to slide within a respective cavity of the drive shaft 125.

Note again that techniques herein are well suited for use in cutting, grinding, etc., types of applications. However, it should be noted that embodiments herein are not limited to use in such applications and that the techniques discussed herein are well suited for other applications as well.

Based on the description set forth herein, numerous specific details have been set forth to provide a thorough understanding of claimed subject matter. However, it will be understood by those skilled in the art that claimed subject matter may be practiced without these specific details. In other instances, methods, apparatuses, systems, etc., that would be known by one of ordinary skill have not been described in detail so as not to obscure claimed subject matter. Some portions of the detailed description have been presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm as described herein, and generally, is considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has been convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a computing platform, such as a computer or a similar electronic computing device, that manipulates or transforms data represented as physical electronic or magnetic quantities within memories, registers, or other information storage devices, transmission devices, or display devices of the computing platform.

While this invention has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting. Rather, any limitations to the invention are presented in the following claims.

What is claimed is:

1. A hand tool comprising:
a handle, the handle disposed at a proximal end of the hand tool;
a cavity, the cavity disposed at a distal end of the hand tool; and
a drive adapter, the drive adapter disposed in the cavity to rotate about an axis of the hand tool, the drive adapter including a hollowed volume extending through the drive adapter from a first opening at a tip of the drive adapter to a second opening of the drive adapter into the cavity.

2. The hand tool as in claim 1, wherein the second opening is disposed on a sidewall of the drive adapter.

3. The hand tool as in claim 1, wherein the drive adapter includes a base end disposed opposite the tip, the base end of the drive adapter coupled to a drive component in the hand tool, the drive component applying a rotational force to the base end of the drive adapter to rotate the drive adapter about an axis.

4. The hand tool as in claim 3, wherein the drive adapter is spring-loaded in the drive component to permit slidable movement of the drive adapter along the axis.

5. The hand tool as in claim 1 further comprising:
a drive component, the drive adapter coupled to a drive source via the drive component, the drive source rotating the drive component about an axis, the drive adapter anchored to the drive component to prevent removal of the drive adapter from the cavity.

6. The hand tool as in claim 5, wherein the first opening at the tip of the drive adapter includes a keyed interface in which to engage a tube of a rotatable blade assembly to the drive adapter, the drive adapter transferring torque from the drive source to the tube of the rotatable blade assembly.

7. The hand tool as in claim 6, wherein the hollowed volume of the drive adapter includes a first hollowed volume portion and a second hollowed volume portion, the first hollowed volume portion extending along the axis from the first opening at the tip of the drive adapter to the second hollowed volume portion, the second hollowed volume portion extending through the drive adapter orthogonal with respect to the axis.

8. The hand tool as in claim 7 further comprising:
a port through which to apply suction to the cavity, the suction drawing matter through a path including: i) a core of the rotatable blade assembly, ii) the first hollowed volume portion, iii) the second hollowed volume portion, iv) the cavity, and v) the port.

9. The hand tool as in claim 8 further comprising:
a seal, the seal disposed between a circumferential outer surface of the drive component and a body of the hand tool, the seal preventing exposure of matter present in the cavity to a bearing resource facilitating rotation of the drive component in the hand tool.

10. The hand tool as in claim 1 further comprising:
a rotatable blade assembly secured to the distal end of the hand tool, the drive adapter providing torque to spin an inner tube disposed in an outer tube of the rotatable blade assembly.

11. The hand tool as in claim 1, wherein the hollowed volume extends from the tip of the drive adapter to multiple openings disposed on a respective sidewall of the drive adapter.

12. The hand tool as in claim 11, wherein the respective sidewall of the drive adapter including the multiple openings is free from contacting a circumferential surface in the hand tool defining the cavity.

13. A drive adapter comprising:
a first opening disposed at a distal end of the drive adapter;
a second opening disposed on a sidewall of the drive adapter, a hollowed volume in the drive adapter extending between the first opening and the second opening; and
a base end of the drive adapter fabricated to securely couple the drive adapter to a drive component.

14. The drive adapter as in claim 13 further comprising:
a keyed region disposed along an axial length of the drive adapter, the keyed region configured to engage the drive adapter to the drive component, the keyed region located along the axial length of the drive adapter between the hollowed volume and the base end.

15. The drive adapter as in claim 13, wherein the keyed region facilitates axial sliding of the base end of the drive adapter with respect to the drive component; and
wherein the keyed region matably connects to the drive component such that the drive adapter and the drive component rotate in unison.

16. The drive adapter as in claim 15, wherein the first opening includes a keyed interface to accept a tube of a blade assembly.

17. The drive adapter as in claim 13, wherein the base end of the drive adapter includes a notch, a retainer resource in the notch securing the base end of the drive adapter to the drive component.

18. A method of fabricating a hand tool, the method comprising:
receiving a drive adapter, the drive adapter including a tip end and a base end, the drive adapter including a hollowed volume extending from the first opening at the tip end through the drive adapter to a second opening on a sidewall of the drive adapter; and
inserting a base end of the drive adapter into a drive component of the hand tool, insertion of the base end securing the drive adapter to the drive component, the tip end of the drive adapter residing within a cavity of the hand tool.

19. The method as in claim 18, wherein inserting the base end of the drive adapter into the drive component of the hand tool further comprises:
anchoring the base end of the drive adapter to the drive component.

20. The method as in claim 18, wherein inserting the base end of the drive adapter into the drive component of the hand tool further comprises:
installing the drive adapter into a cavity at a distal end of the hand tool, the sidewall of the drive adapter rotatably free from contacting an internal surface of the cavity.
